(19) United States
(12) Patent Application Publication
(10) Pub. No.: US 2026/0269054 A1
(43) Pub. Date: Sep. 10, 2026

(54) SYSTEM AND METHOD FOR PROVIDING HOSPITAL CUSTOMER RELATIONSHIP MANAGEMENT

(71) Applicant: FLYINGDOCTOR INC., Gyeonggi-do (KR)

(72) Inventor: Woo Jin LEE, Seoul (KR)

(73) Assignee: FLYINGDOCTOR INC., Gyeonggi-do (KR)

(21) Appl. No.: 19/166,021

(22) PCT Filed: Dec. 15, 2023
(86) PCT No.: PCT/KR2023/020773

§ 371 Date: Dec. 10, 2025

(30) Foreign Application Priority Data
Mar. 17, 2023 (KR) 10-2023-0035035
Oct. 12, 2023 (KR) 10-2023-0135991
Dec. 15, 2023 (KR) 10-2023-0182760

(51) Int. Cl. G16H 40/20; G06Q 30/01; G16H 10/60; G16H 40/67

(52) U.S. Cl. CPC G16H 40/20; G06Q 30/01; G16H 10/60; G16H 40/67

(57) ABSTRACT

A method and apparatus for transmitting CRM content from a medical institution client side assigned to a hospital and a doctor are provided. CRM transmission is performed when CRM content to be transmitted is selected from the medical institution client side and an input of identification information regarding a recipient who is to receive the CRM content is received. On the medical institution client side, a plurality of preset CRM templates are provided as selectable options, and from among same, CRM content transmitted may be selected from among the options through user interfacing on the medical institution client side.

### TECHNICAL FIELD

The following description relates to a system and method for providing customer relationship management (CRM).

### BACKGROUND ART

Customer relationship management (CRM) is the activity of managing communication with customers for personalized marketing. Nowadays, hospitals and clinics (hereinafter referred to as ‘hospitals’) are also practicing CRM by providing information to their customers, or patients, via text messages or SNS, and collecting and processing customer inquiries, requests, and claims for use in patient management. Such CRM in hospitals is also used as a management tool to increase patient satisfaction through proactive communication, ultimately to increase patient visit rates and revisit rates.

The CRM in hospitals typically involves hospital staff contacting patients via phone, email, or text message at necessary times, outsourcing bulk SMS/MMS texting, or sending app-based push notifications or instant messages (e.g., KakaoTalk), most of which remain one-way, one-time messages.

Meanwhile, telemedicine utilizes telecommunications and information technology to provide clinical healthcare over long distances. It eliminates distance barriers and facilitates easier access to medical services. In many cases, telemedicine is used in rural areas where distance makes it difficult to receive ongoing medical services. It is also used to save lives in critical care situations or emergencies. For infectious disease prevention, it is permitted under limited conditions, and recently, telemedicine has been allowed for limited purposes such as medical consultation for patients with chronic conditions or follow-up patients.

(Patent Document 0001) Korean Patent Application Publication No. 2019-0135692 (published on Dec. 9, 2019) proposes a hospital customer relationship management (CRM) system that extracts and processes information necessary for a patient, using medical electronic medical record (EMR) data, to transmit to a client in a message format or provide to a hospital terminal.

(Patent Document 0002) Korean Patent Publication No. 10-1250462 (published on Apr. 8, 2013) proposes a hospital CRM system that provides consultation services for existing patients based on EMR of the existing patients and that facilitates identifying marketing targets, that is, potential medical service users, and actively attracting new patient clients.

(Patent Document 0003) Korean Patent Publication No. 10-0844543 (published on July 8) discloses “Method and Apparatus for Implementing a Web-based remote Diagnosis.”

(Patent Document 0004) Korean Patent Publication No. 10-0366816 (published on Jan. 9, 2003) discloses “Telemedicine Apparatus and Method Thereof.”

### DISCLOSURE OF THE INVENTION

### Technical Goals

A customer relationship management (CRM) provision system is proposed which provides a patient with a communication channel with a hospital and helps the hospital assertively and actively manage the patient and potential patients by linking the hospital to the patient.

A CRM service is provided which is optimized for a hospital that continuously provides an in-advance, on-the-spot, and follow-up care for a patient according to a medical consultation and procedure schedule of the patient.

Particularly, the provided CRM ensures provision of a pre-configured set for each of diseases of a patient and/or procedures to be conducted on a patient so that a medical practitioner/hospital conveniently provides CRM and a patient obtains information in a timely manner.

Furthermore, content with limitations to be delivered through a plain message (SMS or MMS) may be configured to be more comprehensible with a picture/image, contributing to enhancing a patient's satisfaction with the hospital and the completeness of medical consultation in the whole cycle.

A CRM provision system is provided which is excellent in providing various supplementary information and interworking with a hospital EMR, thereby supporting healthcare quality and hospital management.

In addition, enhanced convenience is provided to make editing, customizing, transactions, sharing, and the like of CRM itself easier and more valuable.

### Technical Solutions

### System Configuration and CRM Transmission

A computer-implemented method: A computer-implemented method is provided, which includes receiving, from a medical institution client side assigned to a hospital or a doctor, a selection of customer relationship management (CRM) content to be transmitted and an input of identification information of a recipient to receive the CRM content, and transmitting, in response to the input, the CRM content to a terminal of the recipient. The computer may be an electronic device including at least one processor, a communication interface, and a storage. Such a computer is communicatively connected with a patient-side terminal and a medical institution (or a hospital)-side terminal to provide a service of transmitting CRM content from a client of the medical institution-side terminal to the patient-side terminal.

According to an embodiment, a plurality of preset CRM templates is provided as selectable options on the medical institution client side. In this case, CRM content to be transmitted may be selected from among the options through user interfacing on the medical institution client side.

According to an embodiment, the CRM content includes different transmission dates and times and groups of content to be provided on each of the dates and times. The groups may be understood as a bundle or set of scheduled transmission, which is a sequence of CRM content prepared in advance to be transmitted once.

### Interworking or Integration with Hospital EMR

A medical institution (or a hospital) client interworks with, or is integrated at least in part with, for example, electronic medical record (EMR), or electronic chart, software used in a hospital. However, embodiments are not limited thereto. In this embodiment, at least a portion of identification information of a recipient of CRM to be transmitted may be extracted from the EMR software. The extraction may be automatically performed at the point of ending medical consultation from an EMR medical consultation card that is opened for the medical consultation. Alternatively, the extraction may be selected by searching with a patient name, (part of) phone number, disease name, and the like. In addition, the target recipient of the CRM does not necessarily have to be a single individual but may be multiple individuals depending on the embodiment.

### Generating New CRM or Editing Generated CRM

According to an embodiment, a CRM provision device conveniently provides generation of new CRM content or editing of existing CRM content through a medical institution client. The CRM content to be transmitted includes text and at least one parameter inserted into the text. For example, the parameter includes {patient name}, {appointment date/time}, {hospital name}, {attending doctor}, and the like, but is not limited thereto. When a hospital client is signed in, {hospital name} and/or {attending doctor} may be filled in with default values for the corresponding account. This is optional, of course, and default input may be disabled, or input values may be modified.

When configuring the CRM content to be transmitted as text, a user may insert such parameters, which need to be changed each time, in the form of {parameter} and may separately call and use the {parameter}, which is meta information. The device calls the {parameter} and returns it as the CRM content before actually transmitting the CRM content. Thus, it is transmitted to a patient, who is the recipient, in a natural CRM content format.

According to an embodiment, an edit interface is provided, which allows generating new CRM, or loading or selecting and editing previously generated CRM, in the medical institution client. A configuration of text to be transmitted may be freely modified, a position of {parameter} may also be freely modified, added, or deleted. Staff or a doctor of the hospital may make CRM content, which may include a set of multiple messages, into content customized for the hospital and the patient by adding or deleting a transmission date thereof via the edit interface. Also, such customized content may be saved for future reuse.

### Guidance on Remote Medical Consultation Included in CRM

According to an embodiment, CRM to be transmitted at a date/time that meets legal conditions, in accordance with guidance permitted by law, may optionally include guidance on remote medical consultation and/or a hyperlink for applying for remote medical consultation.

For example, when a record of medical consultation of the recipient at the hospital is confirmed within a predetermined period calculated backwards from the time of transmission and accordingly follow-up remote medical consultation is allowed at the time of transmitting the CRM message, an application (e.g., a link for applying for telemedicine) for the telemedicine provided by the medical institution client is provided being included in the CRM content.

In addition, in the cases in which conditions for follow-up medical consultation are met, guidance on remote medical consultation and an application link may be provided even in the case of D+Day CRM, which is provided for follow-ups on dates after medical consultation or procedure, other than the case of CRM provided in advance in a D-Day format for a hospital appointment date. For example, such guidance on remote medical consultation and an application link may be excluded at timepoints, among CRM content configurations such as a day after (D+1) or three days after (D+3) a date of medical consultation or procedure (D), when follow-up medical consultation is not allowed (e.g., when more than 30 days passed after the last face-to-face medical consultation).

### Conflict Handling During CRM Generation or Editing Process

According to an embodiment, a CRM conflict may be automatically detected and a conflict issue may be handled by a user's selection while generating or editing first CRM or before transmission. For example, when at least one of CRM content and transmission dates/times (e.g., three days before medical consultation) of the first CRM conflicts with second CRM content preset for the recipient, an editing feature is provided to delete at least one of the conflicting items. Such a conflict may be a conflict between CRM messages, a conflict between first medical appointment and second medical appointment that are unnecessary or interfering with each other, or both.

A warning pop-up window may be exposed to the user, for example, a doctor client, and the conflict issue may be resolved immediately according to an input to the pop-up window to modify or delete any one of the conflicting CRMs or medical appointments. According to an embodiment, a modification made during such a conflict issue resolving process may interwork with EMR software used in the hospital, reflecting a change to a medical appointment in the EMR.

### Effects of the Invention

According to the embodiments, the hospital actively connects with patients, enabling patients to receive managed care while staying connected to the hospital. Patients, as customers, are satisfied with the mutual communication with the hospital. The hospital, by managing its customers, can enhance patient safety, patient satisfaction with the hospital, and revisit rates.

By continuously caring for patients according to their medical consultation and procedure schedules, such as before, on the day of, and after, the hospital provides an optimized CRM service.

Pre-configured sets are operated based on the patient's condition and/or the procedure they will undergo. This makes it easier for the medical practitioner/hospital offering CRM and ensures patients receive timely information, enhancing convenience and satisfaction with the medical consultation.

The diverse information and functions included in the CRM contribute to enhancing a patient's satisfaction with the hospital and the completeness of medical consultation in the whole cycle. In addition, various other effects are provided by the embodiments, which will be further described throughout the specification along with explanations of the embodiments.

### BRIEF DESCRIPTION OF DRAWINGS

FIG. 1 is a diagram illustrating a connection between a customer relationship management (CRM) provision device, a patient terminal, and a doctor terminal.

FIG. 2 illustrates a CRM provision device according to an embodiment.

FIG. 3 is a diagram illustrating a screen configuration for generating CRM according to an embodiment.

FIG. 4 illustrates a state in which a CRM template, generated according to an embodiment, is selected and loaded.

FIGS. 5 to 9 are reference screens illustrating various processes for generating and editing CRM.

FIG. 10 illustrates CRM transmitted to a patient terminal, according to an embodiment.

FIG. 11 illustrates a telemedicine service being provided, according to an embodiment.

FIG. 12 is a flowchart illustrating interworking of a CRM provision device with a hospital electronic medical record (EMR).

FIG. 13 is a flowchart illustrating a process in which appointment change and reflection are conveniently integrated within a CRM provision service, according to an embodiment.

FIG. 14 is an example screen illustrating handling of an appointment and/or CRM conflict issue according to an embodiment.

FIG. 15 illustrates CRM content templates provided to a medical institution client, according to an embodiment.

FIG. 16 is a diagram illustrating an ecosystem in which CRM content templates are transacted.

### BEST MODE FOR CARRYING OUT THE INVENTION

Hereinafter, embodiments are described in detail with reference to the accompanying drawings. However, the scope of the right should not be construed as limited by the embodiments. In the drawings, like reference numerals are used for like elements.

The terms used herein are selected from terms generally understood by those skilled in the related art but may have different meanings according to technical developments and/or changes, practices, and preferences of an engineer. Accordingly, the terms used herein should not be construed as limiting the technical spirit and should be construed as illustrative terms to describe embodiments.

In addition, in particular cases, the terms are discretionally selected by the applicant of the disclosure, and the meaning of those terms may be described in detail in the corresponding part of the description. Therefore, the terms used in the disclosure should be understood not based on the terms themselves but based on the meanings of the terms and the content of the disclosure.

A customer relationship management (CRM) provision device may be an electronic device. The electronic device may include general-purpose computing devices implemented by application software and/or system-on-chip (SoC)-type hardware, which provide a service connection and implementation.

The electronic device may include at least one of, for example, a smartphone, a tablet personal computer (PC), a mobile phone, a video phone, an e-book reader, a desktop PC, a laptop PC, a netbook computer, a personal digital assistant (PDA), a portable multimedia player (PMP), an MP3 player, a mobile medical device, a camera, or a wearable device (e.g., a head-mounted device (HMD) such as electronic glasses, electronic clothing, an electronic bracelet, an electronic necklace, electronic appcessory, an electronic tattoo, or a smartwatch).

According to additional embodiments, the electronic device may include at least one of various medical devices (e.g., magnetic resonance angiography (MRA), magnetic resonance imaging (MRI), computed tomography (CT), an imaging device, and an ultrasound device), a navigation device, a global positioning system (GPS) receiver, an event data recorder (EDR), a flight data recorder (FDR), a vehicle infotainment device, an electronic device for vessel (e.g., a marine navigation system and a gyro compass), avionics, a security device, a vehicle head unit, an industrial or household robot, an automatic teller's machine (ATM) of a financial institution, or a point of sales (POS) of a hospital.

In addition, an electronic terminal may include at least one of a portion of a building/structure or smart furniture, an electronic board, an electronic signature receiving device, a projector, or various measuring instruments (e.g., water, electricity, gas, or radio wave measuring instruments) for a telemedicine service connection. The electronic device according to various embodiments of the present disclosure may be one of the various devices described above or a combination thereof. In addition, the electronic device according to various embodiments of the present disclosure may be a flexible device. It is obvious to one of ordinary skill in the art that the type of the electronic device may vary and that the type and main purpose (other than a telemedicine service provision function) of the electronic device are not limited to the above description. Hereinafter, the embodiments are described with reference to the drawings.

### CRM Service Provided by MobiDoc™ According to Embodiments

MobiDoc™ service provides CRM. MobiDoc™ meets personalized needs of patients and also aims to provide hospital-centered services, with a different focus from existing services. Just as patients search for doctors to treat their illnesses, doctors and hospitals also strive to attract and manage patients suited to the hospitals through various methods.

For example, hospitals and doctors send text messages (for example, information on medical consultation, closed hours, hospital events/promotions, and appointment reminders), send messages via SNS services like KakaoTalk, manage websites introducing the hospitals, and recently, even invest time in blogs and YouTube channels to encourage patients to revisit. However, texting and promotional services are pricey, and there are no services optimized for doctors and hospitals.

MobiDoc™ offers advantages in this regard. When using the CRM functions provided by MobiDoc™, clients on the hospital side (i.e., clients logged in and used by doctors and clients logged in and used by hospital staff) collaborate and complement each other's roles. Thus, when a doctor wants to provide special management to a patient while scheduling a next appointment immediately after completing a medical consultation, the doctor may create or edit a CRM and apply it to the patient, and hospital staff may configure the CRM time-effectively. Accordingly, delays in medical consultation may decrease from a medical practitioner's standpoint, and managing hospitals may be done smoothly at hospitals with limited staff relative to workload or when there are staff shortages due to a temporary surge in patients. In addition, compared to bulk SMS or MMS messaging using a bulk text messaging agent service, detailed customization to individual clients and hospitals may be available, a proactive response to changing circumstances may be possible, and greater cost competitiveness may be ensured.

Furthermore, the CRM of MobiDoc™ includes an appointment creation notification, a notification before an appointment date, an appointment day notification, an inspection guide, directions to the hospital, doctor's instructions, a record/guide for a digital prescription after medical consultation, digital therapy, or medical consultation progress monitoring, a post-medical consultation or post-procedure management guide, a MobiDoc™ feed, which is a doctor's professional column, guidance on application for remote medical consultation, an application link, and the like, for a patient.

### System and Service Configurations

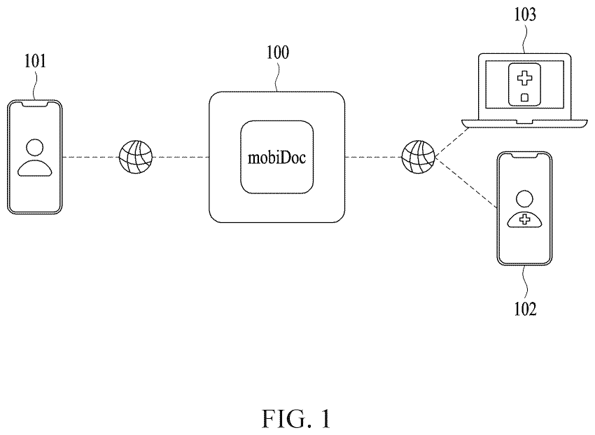

FIG. 1 is a diagram illustrating a connection between a CRM provision device 100, a patient terminal 101, and terminals 102 and 103 belonging to a medical institution.

In the schematic diagram shown, the device 100 is implemented by server-grade computers, workstations, desktop computers, laptop computers, mobile electronic devices, and the like and may also be implemented by a plurality of devices thereamong.

The device 100 is connected to the patient terminal 101 via a network and also connected to the doctor terminal 102 and the hospital terminal 103. Each of the terminals 101 to 103 may correspond to a desktop computer, a laptop computer, and a mobile electronic device such as a smartphone or a tablet PC. Each of the terminals 101 to 103 has a software means referred to as a ‘client’ for convenience.

A patient client is executed on the patient terminal 101. A doctor client is executed on the doctor terminal 102, and a hospital client is executed on the hospital terminal 103, respectively. The doctor client corresponds to a counterpart of the patient client and is installed in a hospital. There may also be an online cloud service type in the form of Software as a Service (SaaS). In this case, the doctor client refers to a service type in which a cloud service is provided when accessed by an account of a doctor and/or hospital staff. The hospital client is a means for hospital administrative staff or nurses, not a medical practitioner (or a doctor) providing medical consultation, to perform medical auxiliary tasks such as making an appointment for medical consultation and receiving hospital fees. The doctor client used by a doctor and the hospital-side client 103 used by hospital staff other than doctors may be provided separately. According to an embodiment, however, the doctor client and the hospital client may be integrated into one, and the entire function may be activated when an account used for sign-in belongs to a doctor who has a doctor's license, and some functions may be disabled when an account used for sign-in belongs to staff other than doctors. The functions to be disabled are those that only doctors should perform, such as ‘starting remote medical consultation.’ Since the timepoint to begin the next patient's medical consultation may be most accurately and appropriately determined by a doctor, the ‘starting medical consultation’ function, which starts a video connection with a patient client waiting in a consultation room, is implemented as a function only executable by the doctor. This is only an example, however, and a medical institution client may be understood throughout this specification to collectively include both the doctor client and the hospital staff client installed on the doctor terminal 102 and the hospital terminal 103, respectively.

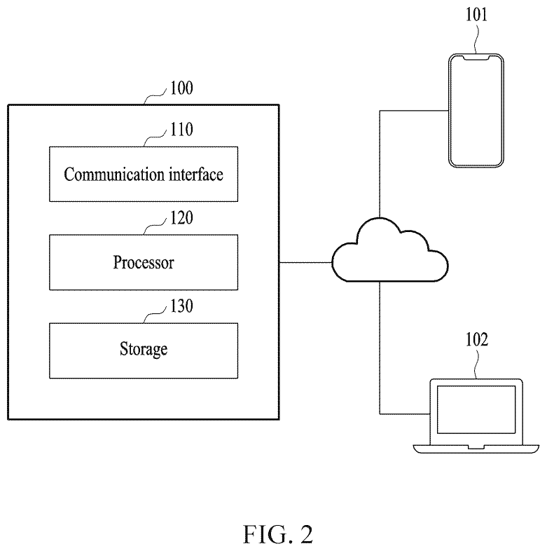

The clients installed on the terminals 101 to 103 may be original application software that is installed, a service provided through a page accessed via a Web-based browser, or a hybrid app having characteristics of an app and the Web. In addition, the clients may be a cloud-based service provided in the form of SaaS. These implementations may be implemented by a developer having ordinary knowledge in this field, so any further detailed description is omitted, and even when an example is mentioned in the descriptions of embodiments, it does not exclude other implementations. An implementation and operation of the device 100 is described with reference to FIG. 2.

### System Configuration and CRM Transmission

FIG. 2 illustrates the CRM provision device 100 according to an embodiment. The device may be an electronic device including at least one processor 120, a communication interface 110, and a storage 130. Such a device is communicatively connected with a patient-side terminal and a medical institution (or a hospital)-side terminal to provide a service of transmitting CRM content from a client of the medical institution-side terminal to the patient-side terminal.

According to an embodiment, a method of providing CRM, provided by the device 100, includes receiving, from a medical institution client side assigned to a hospital or a doctor, a selection of CRM content to be transmitted and an input of identification information of a recipient to receive the CRM content, and transmitting, in response to the input, the CRM content to a terminal of the recipient.

According to an embodiment, a plurality of preset CRM templates is provided as selectable options on the medical institution client side. In this case, CRM content to be transmitted may be selected from among the options through user interfacing on the medical institution client side.

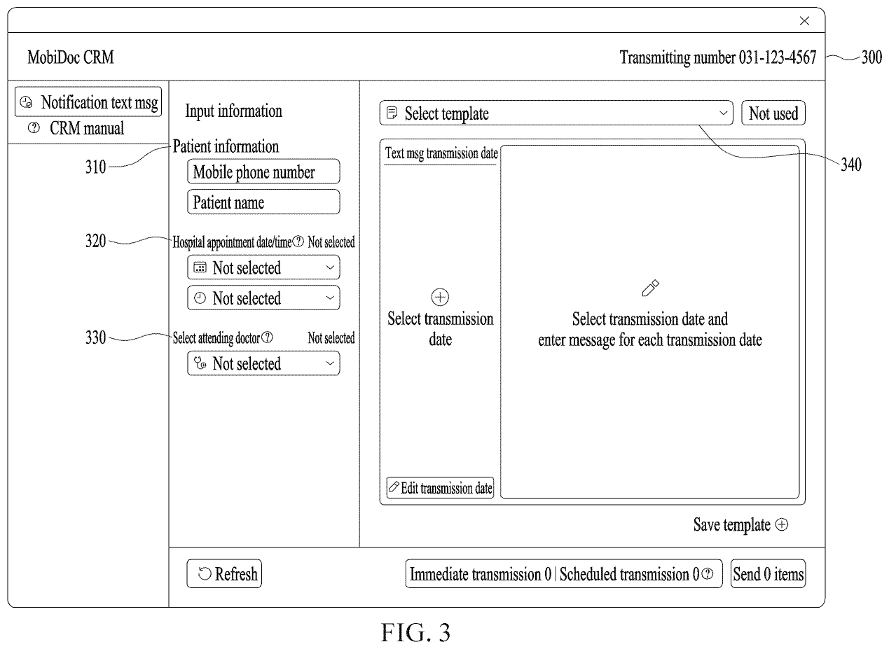

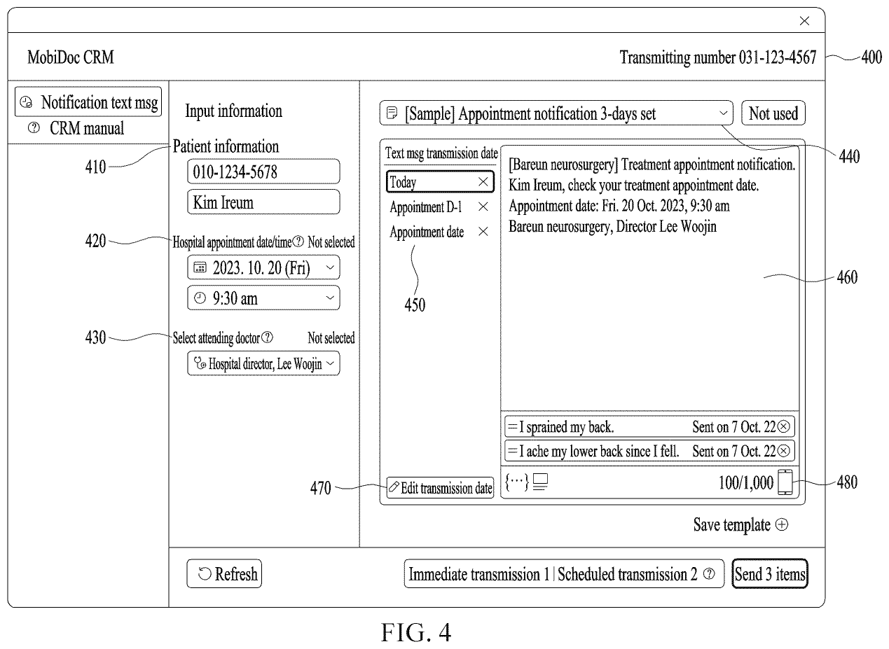

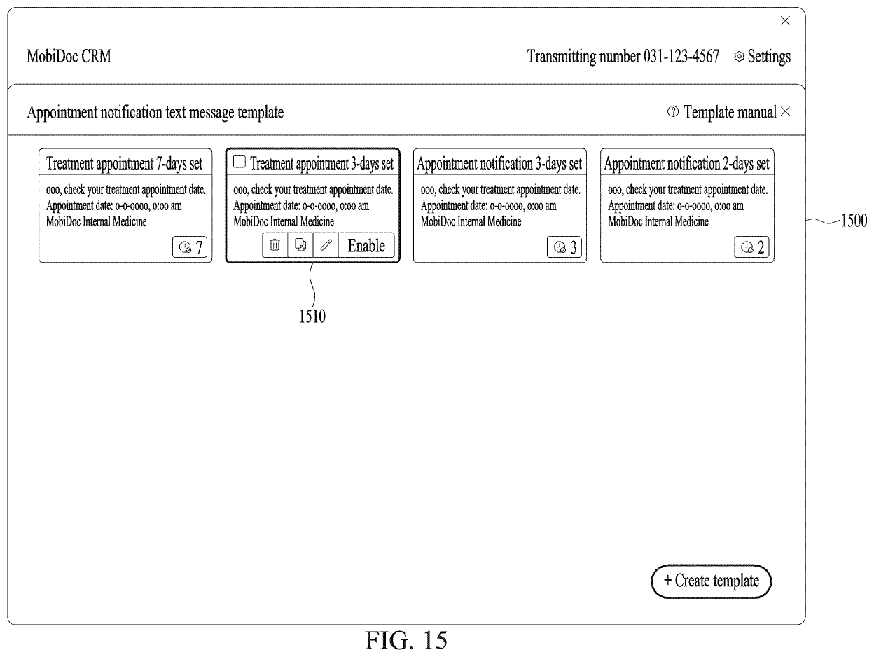

Referring first to FIG. 15, CRM content templates 1500 provided to the medical institution client, according to an embodiment, are illustrated. A set of CRM messages provided for an examination appointment at a total of seven different times, a set of CRM messages 1510 provided for an examination appointment at a total of three different times, and a set of CRM messages provided for a medical appointment at a total of three and two different times are illustrated. A hospital side may manage and use these by generating, editing, deleting, and downloading from MobiDoc™. The actual process of selecting from among the templates and loading or editing CRM are described in detail below with reference to FIGS. 3 and 4.

FIG. 3 is a diagram illustrating a screen configuration 300 for generating CRM according to an embodiment.

The CRM provided by a device provides appointment notification messages, and the most basic function is transmitting an appointment notification message. Referring to FIG. 3, a patient information (a name and a cellphone number) field 310, a target appointment date/time field 320 for transmitting the CRM, a hospital and attending doctor information field 330 are illustrated. The information may include, in addition to those entered by typing in manually, information that the MobiDoc™ CRM retrieves from the EMR of the hospital and may be easily searched and selected by a doctor or a hospital searching for a patient's name or part of a phone number within a CRM section of a doctor client of MobiDoc™. In addition, the information may be patient information that the MobiDoc™ CRM retrieves from the hospital EMR and import to the MobiDoc™ CRM. Templates previously saved may be selected from a template selection field 340 and loaded. According to an embodiment, CRM content includes different transmission dates and times and groups of content to be provided on each of the dates and times. The groups may be understood as a bundle or set of scheduled transmission, which is a sequence of CRM content prepared in advance to be transmitted once.

FIG. 4 illustrates a state 400 in which a CRM template, generated according to an embodiment, is selected and loaded.

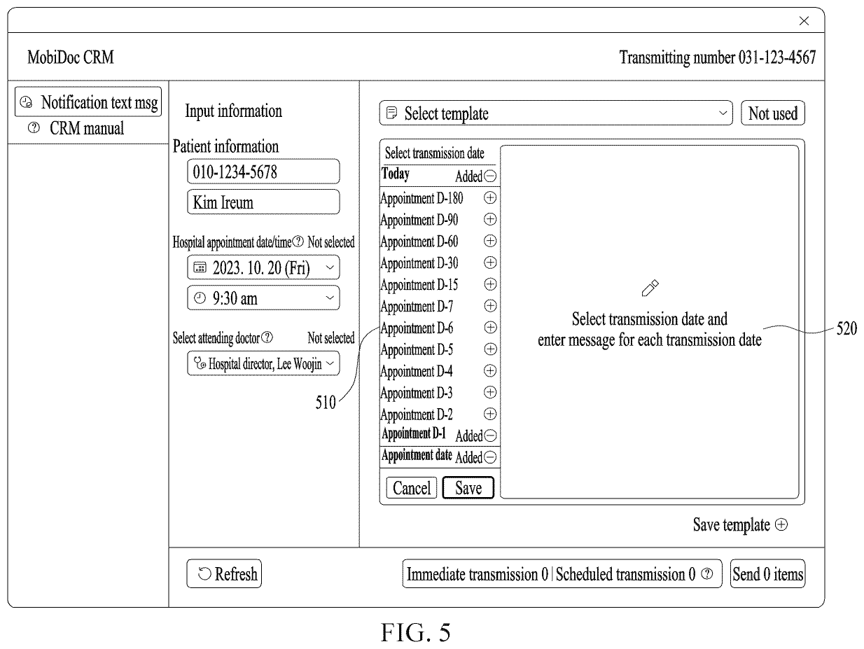

Referring to FIG. 4, a patient information field 410, an appointment date/time field 420, and an attending doctor information field 430 are filled. In addition, an appointment notification three-days set is selected from a template selection field 440 and loaded. A default setting value (e.g., three-times transmission-normally, when making a next appointment after visiting for medical consultation—on the day to make the appointment, one day prior to the appointment (D−1), and the day of the appointment (D-day)) preset by the doctor or hospital may be set. These transmission dates/times are shown in a field 450, and when each one is selected, content corresponding to each date is shown in an editing window 460. When an edit transmission date button 470 is pressed, a screen shown in FIG. 5 is displayed.

Referring to FIG. 5, depending on the type of procedures, a patient's propensity, or circumstances, a doctor may additionally select or delete another day using a toggle button in a temporary selection field 510. A message corresponding to each date may be edited in an editing window 520.

### Generating New CRM or Editing Generated CRM

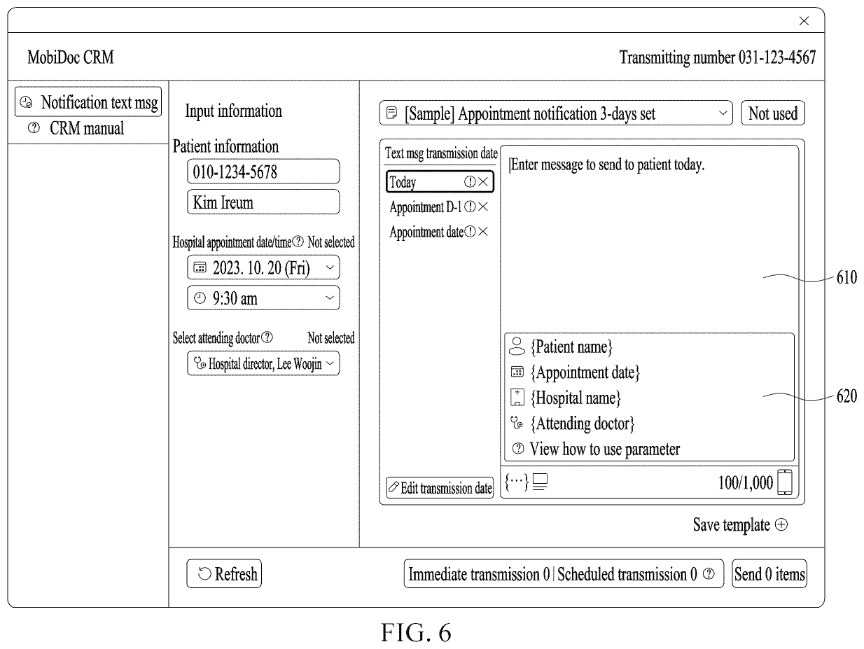

FIGS. 6 to 9 are reference screens illustrating various processes for generating and editing CRM.

According to an embodiment, a CRM provision device conveniently provides generation of new CRM content or editing of existing CRM content through a medical institution client. The CRM content to be transmitted includes text and at least one parameter inserted into the text. For example, the parameter includes {patient name}, {appointment date/time}, {hospital name}, {attending doctor}, and the like, but is not limited thereto. When a hospital client is signed in, {hospital name} and/or {attending doctor} may be filled in with default values for the corresponding account. This is optional, of course, and default input may be disabled, or input values may be modified.

Referring to FIG. 6, parameters are set in a field 620, and a patient name, appointment date/time, hospital name, attending doctor, and the like are provided to the parameters. Basically, the hospital name and attending doctor are filled in from set values of account information of an account currently accessing a MobiDoc™ doctor client, which may be modified by selection. As described above, the patient name/phone number, appointment date/time, and the like may also be filled in by search or input/modified manually.

As described above, content of a message to be transmitted may be manually input in a field 610, or multiple templates may be provided to select a template to be transmitted. Importing from an external clipboard and pasting in the field 610 is also possible. Thereafter, fields of the field 620 may be freely dragged into the field 610 and arranged.

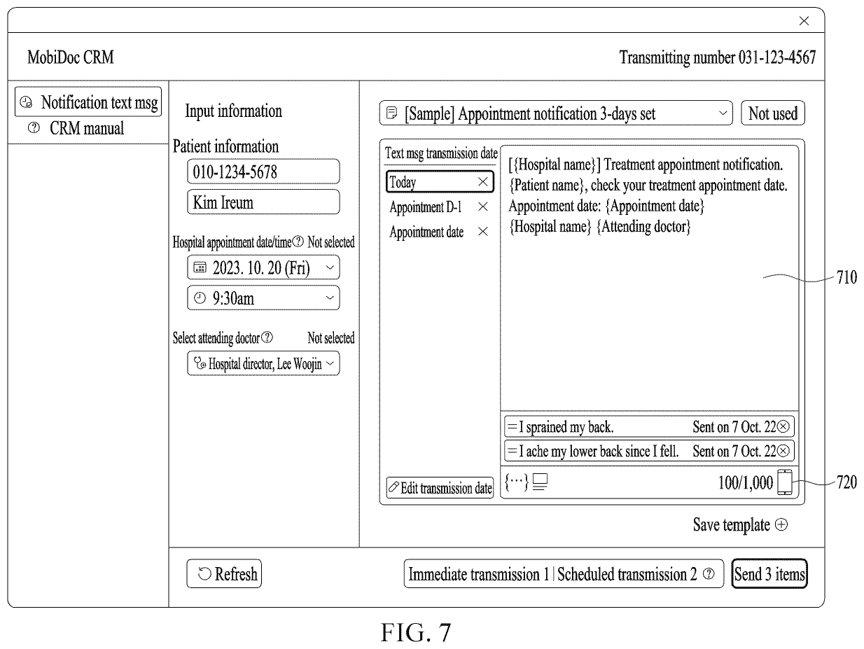

Referring to FIG. 7, in the case of importing from pre-provided templates, when a doctor or hospital staff selects an appointment notification three-days set from among templates provided by the CRM, the template is loaded in displayed in a field 710. Then the values (e.g., a patient name and an appointment date/time) of a {parameter}, which are input or set on the left, may be inserted into the {parameter}s in the template to complete the message. The field 710 is set to an edit mode in FIG. 7, in which the user (e.g., the doctor/hospital staff) may focus on the input field to switch it to the edit mode when necessary. In that case, the {parameter} items may be confirmed as described above with reference to FIG. 6.

As described above, when configuring the CRM content to be transmitted as text, a user may insert such parameters, which need to be changed each time, in the form of {parameter} and may separately call and use the {parameter}, which is meta information. The device calls the {parameter} and returns it as the CRM content before actually transmitting the CRM content. Thus, it is transmitted to a patient, who is the recipient, in a natural CRM content format.

According to an embodiment, an edit interface is provided, which allows generating new CRM, or loading or selecting and editing previously generated CRM, in the medical institution client. A configuration of text to be transmitted may be freely modified, a position of {parameter} may also be freely modified, added, or deleted. Staff or a doctor of the hospital may make CRM content, which may include a set of multiple messages, into content customized for the hospital and the patient by adding or deleting a transmission date thereof via the edit interface. Also, such customized content may be saved for future reuse.

When the user's selection cursor selects an area outside the field 710 for editing or clicks on a preview button 720, a preview is displayed in the form of a completed CRM message with the {parameter}s replaced by ‘parameter values.’ When the preview button 720 is pressed in the current state shown in FIG. 7, the state changes into one such as the field 460 of FIG. 4. When a preview button 480 is pressed again, the state changes into the edit mode.

### Management of Patient after Medical Consultation/Procedure Through CRM (D+Day CRM)

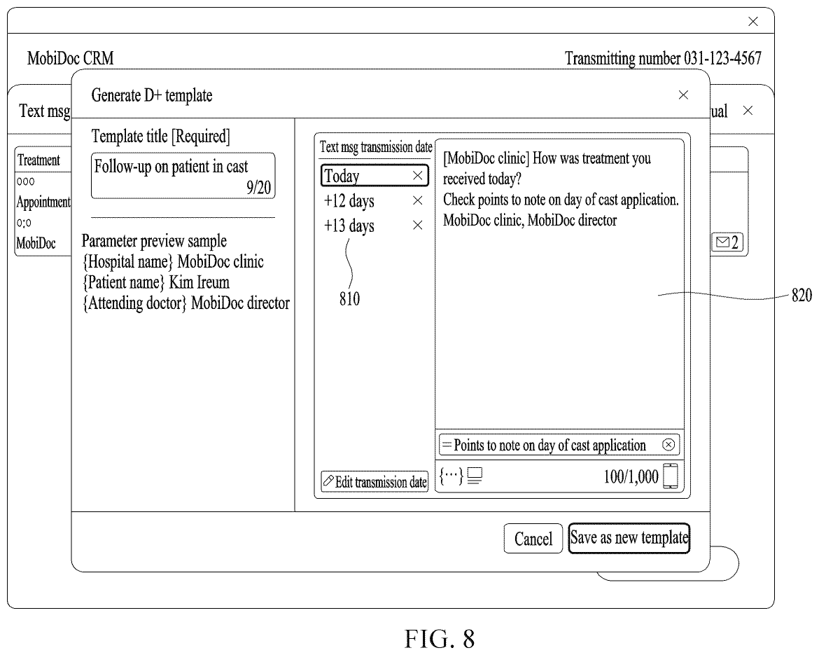

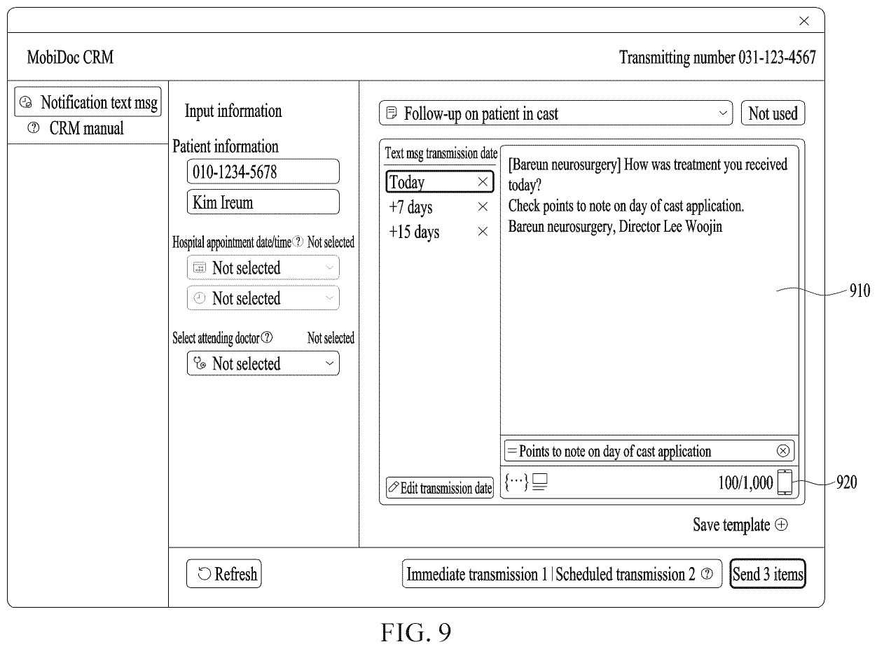

FIGS. 8 and 9 are diagrams illustrating D+Day CRM provided for follow-up management of a patient or information provision after medical consultation or a procedure, rather than D-Day CRM, which sends advance guidance messages.

For example, after a dermatological procedure, follow-up management may need to be scheduled to be done, such as notifying on what actions should be taken and what precautions should be observed a day after the procedure, which information to provide three days after the procedure, and what check-ups to do one week after the procedure and contact the hospital when any issues arise. Conventionally, most dermatology clinics provide printed handouts for these cases, causing various inconveniences. Such needs exist not only for dermatological procedures but also for most cases, such as various surgical operations including plastic surgery, interventional procedures for pain management, cardiac interventional procedures, medical interventions for specific symptoms, and the like. During the COVID-19 pandemic that swept the globe over the past few years, there was also a need to provide infected patients with systematic information and assist health management through date-based guidance. MobiDoc™ CRM is capable of playing a role in this area going forward.

FIG. 8 illustrates a process of generating a D+Day template used in orthopedics. Dates on which a CRM message is to be transmitted according to a selected or generated schedule are shown in a field 810. When one is selected therefrom, a message including text may be loaded together with {parameter}s in a field 820 on the right. A user may edit the message here, and add or delete a date in the field 810 on the left.

When pressing a preview button 920 after editing of the template is completed, a completed form of CRM message is previewed, in which ‘parameter values’ are inserted into {parameter} in the field 910.

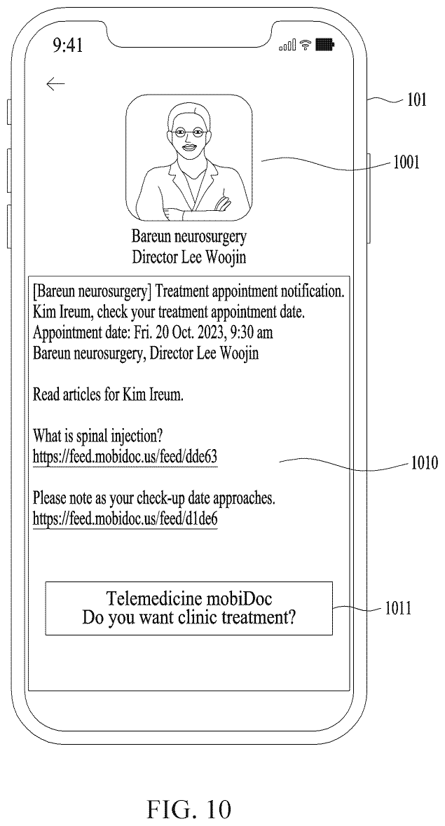

FIG. 10 illustrates CRM transmitted to a patient terminal, according to an embodiment.

On the patient terminal 101, a hospital and doctor information that transmitted the CRM is displayed in an upper field 1001, and a CRM message is provided in a lower field 1010. The layouts of the fields 1001 and 1010 and a field 1011 illustrated in FIG. 10 are only examples. Thus, other sizes and layouts are possible. For example, unlike FIG. 10, it may be configured like a messaging app interface of a smartphone or a direct message (DM) interface of an SNS.

A process or configuring a CRM message and content thereof are the same as described above. Two items not covered above are described below.

### Feed Content Included in CRM

As illustrated in FIG. 10, a CRM message may include information on diseases, health information, and other professional articles sent by a relevant doctor or hospital to a patient. The articles may be directly inserted or presented as hyperlinks as shown. The hyperlinks connect to corresponding MobiDoc™ feed articles on an app and/or the Web. Since the MobiDoc™ feed may only be authored by licensed doctors, content thereof may be considered verified. Thus, when a doctor who treated the patient sends specialized column articles suited for the patient's disease and physical condition by inserting them in the form of feed in the CRM, the patient's attention and focus on the CRM itself are high. As mentioned above, a CRM message in the form of SMS or MMS, which is a traditional form sent from hospitals, may be considered as advertisements (ads) and ignored by patients. However, when a feed is provided as illustrated in the CRM field 1010 of FIG. 10, patients may perceive that the hospital and doctor that sent the feed genuinely treat them as customers and care about them.

Consequently, good functions of CRM may be realized, helping hospitals manage and strengthen relationships with patients, who are their customers.

Such enhancement of the functions of CRM may be further increased by combining with ‘MobiDoc clinic,’ remote medical consultation of MobiDoc™.

### Guidance on Remote Medical Consultation Included in CRM

Even when patients schedule an appointment for next medical consultation (i.e., a follow-up appointment), a no-show rate may be high. Reasons why patients who scheduled a follow-up appointment cancel the appointment or fail to show may vary, including not being sure about the necessity of follow-up medical consultation and/or a difficulty of visiting the hospital due to work, childcare, or the like. MobiDoc clinic, which is remote medical consultation provided by MobiDoc™, functions as a nudge to help those patients easily access the follow-up medical consultation via CRM. By having a remote consultation even for a brief moment on their illness progression, current condition, and side effects of prescribed medications with a doctor who conducted the first visit, patients may determine whether an additional visit to the hospital is necessary, which is a positive effect.

Thus, according to an embodiment, guidance informing that MobiDoc remote medical consultation may be applied for in the field 1011 of FIG. 10 and/or a hyperlink for applying for the remote medical consultation are provided as part of the CRM.

However, the criteria permitting such remote medical consultation may vary by country and time period. Thus, according to an embodiment, CRM to be transmitted at a date/time that meets legal conditions, in accordance with guidance permitted by law, may optionally include guidance on remote medical consultation and/or a hyperlink for applying for remote medical consultation.

For example, it may be configured that the guidance and application link for remote medical consultation are provided in the field 1011 only when the follow-up medical consultation is within a predetermined period calculated backwards from the point of transmitting the illustrated CRM, for example, within 30 days, during which remote medical consultation is allowed. To achieve this, D−0 day (the day of a previous appointment) information in the CRM based on previous appointment information and/or EMR of the hospital may be utilized and compared to a point to subsequently transmit the CRM. That is, it is distinguished whether it is CRM transmitted 7 days after the previous medical consultation or CRM transmitted 2 months after the previous medical consultation. When the criteria permitting remote medical consultation allow only follow-up medical consultation for the same disease at the same hospital within 30 days, the former (CRM transmitted 7 days after) includes the guidance and application link for remote medical consultation, but the latter (CRM transmitted 2 months after) does not guide on this.

In addition, in the case of D+Day CRM, which is for follow-up management of previous medical consultation or procedures, it may also be configured that the guidance and application link for remote medical consultation are provided within a predetermined reference period (e.g., 30 days). For example, such guidance and application link for remote medical consultation may be excluded at timepoints, among CRM content configurations such as a day after (D+1) or three days after (D+3) a date of medical consultation or procedure (D), when follow-up medical consultation is not allowed (e.g., when more than 30 days passed after the last face-to-face medical consultation).

According to another embodiment, CRM may be transmitted before the limit for allowing follow-up medical consultation expires to ask whether the patient wants remote medical consultation for follow-up medical consultation. For example, the guidance and application link for remote medical consultation may be transmitted to a patient in the form of CRM once when 21 days have passed, and once again when 28 days have passed, since the first medical consultation or procedure.

### MobiDoc Clinic (Remote Medical Consultation) Started Via CRM

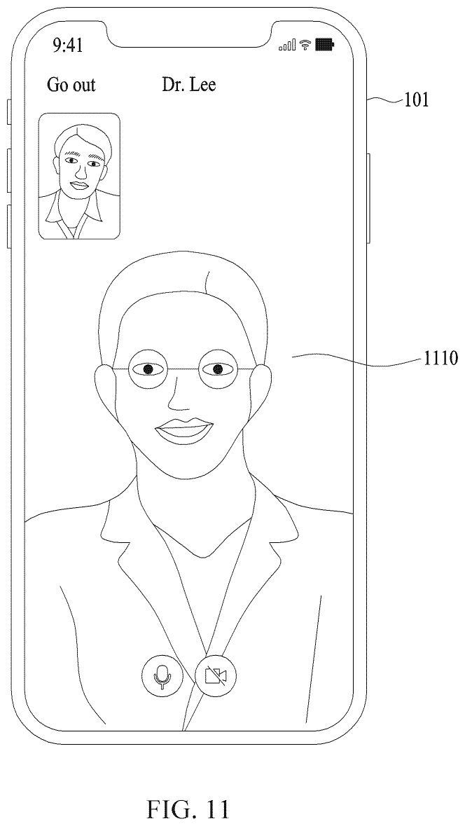

FIG. 11 illustrates remote medical consultation being started and conducted using a link in the field 1011.

Although not shown, a patient client may complete an application for medical consultation and requests access to a consultation room. Then, upon approval of a hospital and/or doctor, a client of the patient's account is admitted to a stage referred to as consultation room. This indicates that a service status for the patient client of the patient's account transitions from a medical consultation application process to a medical consultation waiting process. The consultation room is a stage corresponding to a status in which, when a doctor starts medical consultation, a video and/or voice call between them may start immediately. It is a user experience similar to one in an offline hospital, where a nurse guides a patient into the consultation room to wait in a place for medical consultation. When the nurse notifies a doctor that the patient is ready, the doctor enters whenever ready and starts the medical consultation for the patient. In university hospitals, doctors often move between two connected consultation rooms for medical consultation. While a doctor's medical consultation is in progress in one consultation room, a nurse has a next patient enter a connected consultation room to get ready for medical consultation. Here, information such as a chart, necessary medical images, and inspection results is prepared through an EMR system or other means to prevent the busy doctor from wasting time on such preparatory tasks. This is one of the advantages of MobiDoc clinic™.

This is similar to a large hospital where a doctor moves between two consultation rooms, completing medical consultation of one patient in one consultation room, performing necessary charting, and moving to the other consultation room, in which next medical consultation is prepared and a next patient is also waiting, to immediately start the next medical consultation. This reduces unnecessary waste of time, thereby allowing the doctor to focus on the essence of medical consultation even for the same number of patients, reducing patient inconvenience caused by delayed medical consultation, and providing medical services to more patients. The consultation room’ entry waiting of MobiDoc clinic described above plays this role.

Thus, it ensures patients are kept waiting and guarantees a possibility of an immediate start of medical consultation. Such improvement of medical consultation efficiency provides better service for both medical practitioners and patients. In most of existing telemedicine services, when a patient requests online (or remote) medical consultation, a doctor (or hospital) side attempts to connect with the patient. This connection may be via a video call, voice call and the like. However, since the remote medical consultation is not a face-to-face meeting but an online connection, the patient who was waiting for the remote medical consultation with the doctor may not remain on hold for the medical consultation but engage in other activities or leave the terminal and go elsewhere. Then the possibility of immediate medical consultation may not be guaranteed when the doctor (or hospital) attempts to start the medical consultation.

MobiDoc clinic presented in FIG. 11 checks, through the patient client, whether the patient is actually ready for the medical consultation and thus the medical consultation may immediately start when the doctor starts a medical consultation process. This is confirming that the patient is in a standby status, in which the patient has entered the consultation room, and communicates the status to a doctor client.

After checking the patient client side, when the patient is on the phone, not looking at the screen and has left the spot, or performing another task on the terminal 101 and accordingly, when the doctor may not immediately start medical consultation as attempted but has to wait, the doctor may be notified of the situation. Furthermore, in addition to the notification, the doctor may be reminded that ‘the patient may not be ready for medical consultation’ and given the option to see another patient first. An alert may be sent to the patient indicating ‘Medical consultation may not start until you are ready’ to create a nudge effect to ensure the patient is prepared.

With this consideration, the doctor may know which patient among those who have applied for medical consultation and entered the waiting room is prepared for the start of medical consultation and select a well-prepared patient to start the medical consultation, thereby preventing the doctor and the hospital from experiencing inefficiencies due to connection delays or failures. Hospital staff or a telemedicine service provider side may guide an unprepared patient to be ready for the medical consultation (e.g., by not closing the app, not performing another task, and waiting while monitoring the consultation room screen) by notifying via the patient terminal that the medical consultation may start when the patient is ready. According to the above embodiments, the possibility of immediate medical consultation by the doctor for the patient may be increased.

FIG. 11 illustrates a state 1110 of a patient terminal after medical consultation has started. A patient is informed that follow-up medical consultation is available via remote medical consultation and may receive medical consultation without visiting a hospital in person. This is particularly effective when the necessity of follow-up medical consultation is unclear, since consulting with a doctor on the progression of illness and adjusting medication is possible without having to make time to visit the hospital. Thus, it may serve to help a patient hesitant about follow-up medical consultation continue receiving medical consultation from the doctor without giving up the follow-up medical consultation. Consequently, it may reduce potential gaps in medical consultation that patients might overlook and prevent deterioration of patients' health.

### Interworking or Integration with Hospital EMR

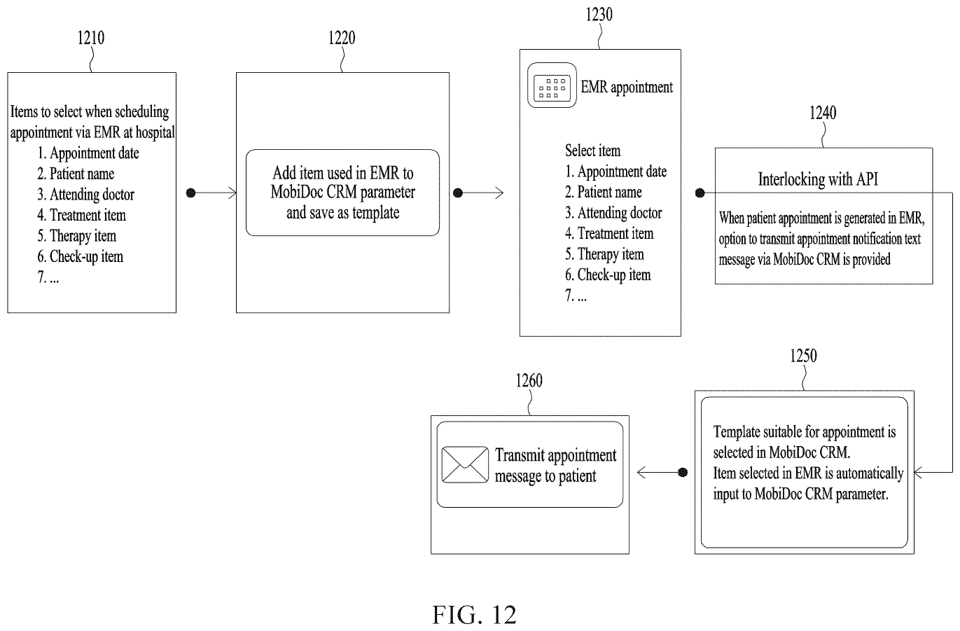

FIG. 12 is a flowchart illustrating interworking of a CRM provision device with a hospital EMR.

A medical institution (or a hospital) client interworks with, or is integrated at least in part with, for example, electronic medical record (EMR), or electronic chart, software used in a hospital. However, embodiments are not limited thereto. In this embodiment, at least a portion of identification information of a recipient of CRM to be transmitted may be extracted from the EMR software. The extraction may be automatically performed at the point of ending medical consultation from an EMR medical consultation card that is opened for the medical consultation. Alternatively, the extraction may be selected by searching with a patient name, (part of) phone number, disease name, and the like. In addition, the target recipient of the CRM does not necessarily have to be a single individual but may be multiple individuals depending on the embodiment.

FIG. 12 presents an example flowchart of a process in which MobiDoc™ CRM is interworking with a hospital EMR. Generally, when making an appointment using EMR at a hospital, an appointment date, patient name, attending doctor, medical consultation item, treatment item, examination item, and the like are selected, as in operation 1210. MobiDoc™ CRM adds items used in the EMR of the corresponding hospital to MobiDoc CRM parameter and saves them as templates, as in operation 1220.

In addition, when an appointment is generated in the EMR, all or a selected portion of the items are utilized for transmitting scheduled text messages of MobiDoc™ CRM, as in operation 1230. Here, MobiDoc™ CRM may be application programming interface (API)-interworking with the EMR as in operation 1240. In this case, when a patient appointment is generated in the EMR, an option to transmit an appointment notification text message via MobiDoc™ CRM is provided. When transmission is selected, MobiDoc™ CRM provides a template suitable for that appointment as in operation 1250, and hospital staff or a doctor may approve the provided template or select another one considered more appropriate from other templates. Once the template to be transmitted is selected, MobiDoc™ CRM imports the parameters of the EMR to generate a text message to be actually transmitted, and the generated text message is transmitted as in operation 1260.

As described above, the interworking between MobiDoc™ CRM and the EMR minimizes time of doctors or hospitals spent in CRM.

### Quick Appointment Change Handling of CRM

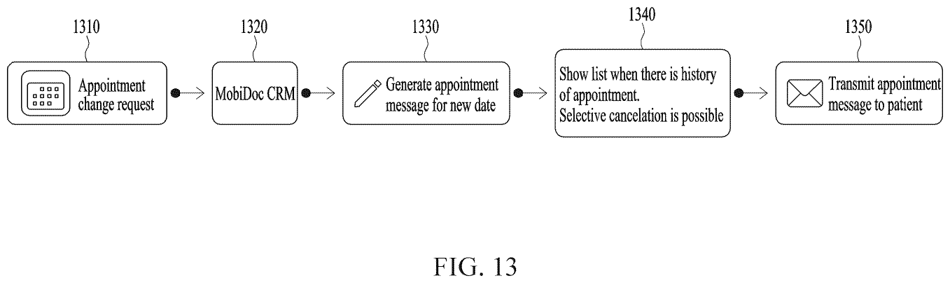

FIG. 13 is a flowchart illustrating a process in which appointment change and reflection are conveniently integrated within a CRM provision service, according to an embodiment. With MobiDoc™ CRM, handling appointment changes is also more convenient compared to existing CRM text message service.

In a typical case where CRM and EMR operate separately, an existing appointment to be canceled or changed and an associated CRM need to be found and canceled, and generating a new appointment message and actually transmitting the new appointment message according to a changed, new appointment needs to be done manually. When this is not handled correctly, it could cause confusion with the previously transmitted text message.

In MobiDoc CRM, however, existing appointments that were scheduled and are to be transmitted may be searched within MobiDoc CRM all at once, so CRM may be selectively canceled/changed/added on a single interface without closing a window. Consequently, handling an appointment change process is streamlined, and integrated management with the original appointment before changing and other CRMs for another medical consultation of that patient is possible at the same time. This is described with reference to FIG. 13.

A process of reflecting an appointment change in MobiDoc™ CRM is illustrated. When an appointment change request 1310 occurs, MobiDoc CRM 1320 is activated, and generating an appointment message for a new date is immediately started in operation 1330. In this case, in operation 1340, a CRM provision device according to an embodiment shows a list when there is a history of transmission of an appointment message to that patient via MobiDoc CRM. When a specific message is selected from the list, content thereof may be reviewed. Doctors of hospital staff may selectively cancel or change some of the list, or add new items. The changed appointment message is immediately transmitted to the patient in operation 1350.

That is, management of transmission of previously generated CRM text messages is integrated with management of new CRM text messages to be generated, enabling unified and one-stop processing without leaving the page. From the patient's perspective, confusion due to receiving incorrect or duplicate CRM text messages is prevented, and the hospital staff's work is also greatly simplified.

### Conflict Handling During CRM Generation or Editing Process

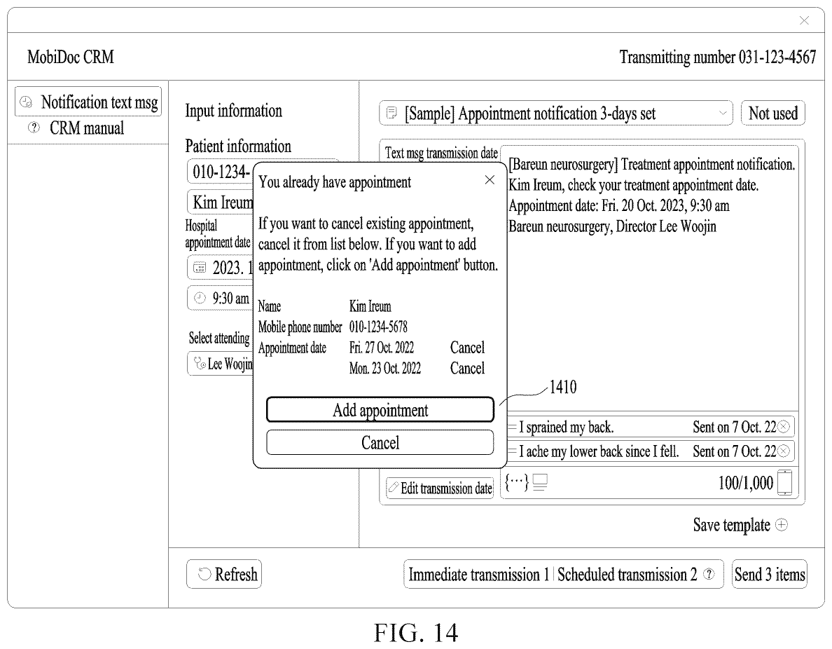

FIG. 14 is an example screen illustrating handling of an appointment and/or CRM conflict issue according to an embodiment.

A CRM conflict may be automatically detected and a conflict issue may be handled by a user's selection while generating or editing first CRM or before transmission. For example, when at least one of CRM content and transmission dates/times (e.g., three days before medical consultation) of the first CRM conflicts with second CRM content preset for the recipient, an editing feature is provided to delete at least one of the conflicting items. Such a conflict may be a conflict between CRM messages, a conflict between first medical appointment and second medical appointment that are unnecessary or interfering with each other, or both.

A warning pop-up window 1410 may be exposed to the user, for example, a doctor client, and the conflict issue may be resolved immediately according to an input to the pop-up window 1410 to modify or delete any one of the conflicting CRMs or medical appointments. According to an embodiment, a modification made during such a conflict issue resolving process may interwork with EMR software used in the hospital, reflecting a change to a medical appointment in the EMR.

### CRM Template and Template Transaction Store

FIG. 15 shows a screen 1500 on which MobiDoc CRM templates are managed. Templates are saved and managed individually for each hospital or each user. Here, a determined CRM template 1510 may be selected for editing, such as using for CRM transmission, sharing with others, or deleting from a template pool.

While MobiDoc provides MobiDoc™ CRM text message templates by default, member hospitals may create various templates optimized for the hospitals or suitable for the type and circumstances of medical consultation/procedures for diseases. For medical examinations or disease-specific tests, a set to notify patients from 7 days in advance with detailed guidance/instructions on the type or scope thereof and a set to notify patients from 3 days in advance are available. Each set may include multiple CRM text messages pre-configured with {parameter} and content. The same applies to appointment notifications. Furthermore, notifications may be set based on time even within a day.

In addition, different templates may be prepared or generated and managed for each patient. Templates may be generated and managed for various circumstances, such as for patients with limited mobility, patients residing in other regions who come for medical consultation from a distant region, patients who have to stop taking medications, which they regularly take, days before a certain examination, and the like. This is the know-how of the hospitals and the competitiveness of the MobiDoc template service.

Well-generated and well-managed CRM content templates may be shared or transacted, and an ecosystem may be created within a market store of MobiDoc. Users may generate good templates and share them with MobiDoc's hospital members, and the well-generated templates may be transacted between hospitals. In addition, MobiDoc's patient clients may generate CRM templates they need and propose them to hospitals they choose for implementation. Furthermore, patients may also have an agreement with the hospital that they possess their own templates and have the templates transmitted to them after medical consultation.

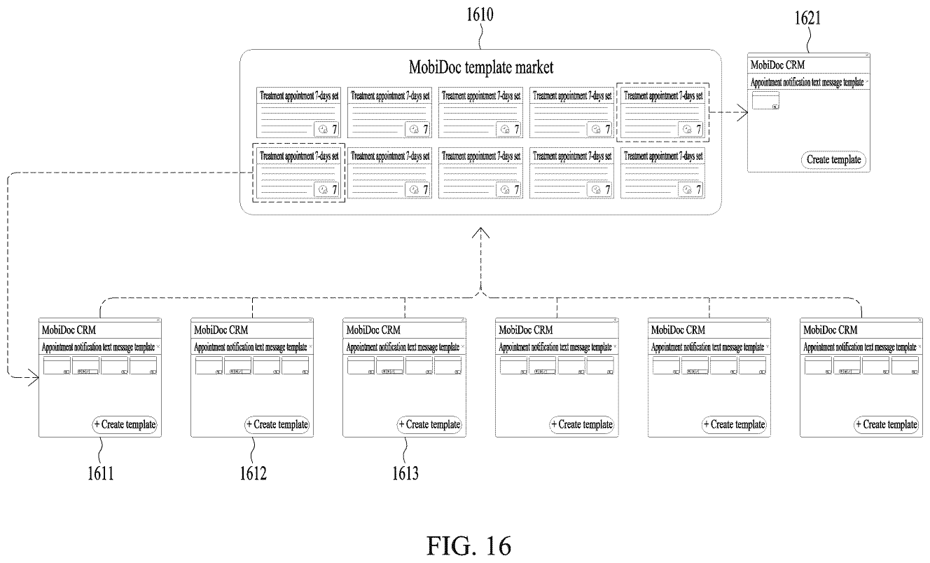

FIG. 16 is a diagram illustrating an ecosystem in which CRM content templates are transacted.

Similar to MobiDoc™ CRM text message templates on App Store/Play Store, the templates may be transacted in units of templates 1611, 1612, 1613, and the like on a MobiDoc CRM market 1610. When a hospital generates a template by optimizing, the hospital may register the template on the MobiDoc CRM market 1610, and other hospitals may use the template, as in operation 1621. Other than hospitals, any other companies or experts with expertise in CRM may design CRM templates and upload them on the MobiDoc market for member hospitals to use.

The MobiDoc template store 1610 itself may be continuously improved to enhance customer management services of hospitals. Furthermore, templates may be transacted to reward hospitals or individuals that contributed to the improvement of productivity. In addition, a bill may be charged in proportion to template usage, and sharing templates among groups (e.g., affiliated/collaborating hospitals) is also possible. MobiDoc™ may provide continuously evolving and user-participating ecosystem, contributing to open development in CRM services.

The embodiments described herein may be implemented using a hardware component, a software component, and/or a combination thereof. The devices and components described in the embodiments may be implemented using a general-purpose computer or a special-purpose computer, such as, for example, a processor, a controller, an arithmetic logic unit (ALU), a digital signal processor (DSP), a microcomputer, a field-programmable gate array (FPGA), a programmable logic unit (PLU), a microprocessor, or any other device capable of responding to and executing instructions. A processing device may run an operating system (OS) and one or more software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. In addition, a different processing configuration is possible, such as one including parallel processors.

The software may include a computer program, a piece of code, instructions, or one or more combinations thereof, to independently or collectively instruct or configure the processing device to operate as desired. Software and/or data may be embodied permanently or temporarily in any type of machine, component, physical or virtual equipment, computer storage medium or device, or in a propagated signal wave for the purpose of being interpreted by the processing device or providing instructions or data to the processing device. The software may also be distributed over network-coupled computer systems so that the software is stored and executed in a distributed fashion. The software and data may be stored by one or more non-transitory computer-readable recording mediums.

The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the purposes of embodiments, or they may be of the kind well-known and available to one of ordinary skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as read-only memory (ROM), random-access memory (RAM), flash memory, and the like. Examples of program instructions include both machine code, such as those produced by a compiler, and files containing high-level code that may be executed by the computer using an interpreter. The above-described hardware devices may be configured to act as one or more software modules in order to perform the operations of the embodiments, or vice versa.

Although the embodiments have been described with reference to the limited number of drawings, it will be apparent to one of ordinary skill in the art that various modifications and variations may be made to the embodiments. For example, suitable results may be achieved if the described techniques are performed in a different order and/or if components in a described system, architecture, device, or circuit are combined in a different manner and/or replaced or substituted by other components or their equivalents.

Therefore, other implementations, other embodiments, and equivalents to the claims are also within the scope of the following claims.

1. A computer-implemented method comprising:
receiving, from a medical institution client side assigned to a hospital or a doctor, a selection of a first group of customer relationship management (CRM) contents to be transmitted and an input of identification information of a recipient to receive the CRM content; and
transmitting, in response to the input, the first group of CRM contents to a terminal of the recipient,
wherein the first group of CRM contents is a sequence determined based on the selection and including different transmission dates and times and a group of contents to be provided to the recipient at each of the dates and times,
wherein contents included in the first group of CRM contents include different information for each date and time according to a procedure to be performed on the recipient, a propensity of the recipient, or circumstances,
wherein the receiving of the input of the identification information comprises:
providing a plurality of preset CRM templates as selectable options on the medical institution client side;
providing, on the medical institution client side, a user interface that separately displays a series of contents to be sent to each patient by date within the first group of CRM contents;
providing, on the medical institution client side, an editing feature to delete at least one of conflicting items when at least one of a content and transmission date and time of the first group of CRM contents conflicts with a content and a transmission date and time of a second group of CRM contents preset for the recipient;
receiving, from the medical institution client side, a selection of a plurality of different dates and times for the first group of CRM contents and a plurality of contents to be provided to the recipient at each of the plurality of dates and times; and
determining contents to be included in the first group of CRM contents based on the selection received from the medical institution client side,
wherein, on the medical institution client side, a content corresponding to each of the separately displayed dates and times may be edited and a date and time for providing a content may be added or deleted through the user interface; and
the transmitting of the first group of CRM content comprises:
transmitting, to the terminal of the recipient, content selected to be provided at a corresponding date and time among the group of contents when each of the transmission dates and times arrives.

2. (canceled)

3. (canceled)

4. The computer-implemented method of claim 1, wherein
the medical institution client interworks or is integrated with electronic medical record (EMR) software used in the hospital, and at least a portion of the identification information of the recipient is extracted from the EMR software.

5. The computer-implemented method of claim 1, wherein
a CRM content of the first group of CRM contents to be transmitted comprises text and at least one parameter to be inserted into the text, and
the input identification information of the recipient is returned to the parameter so that the CRM content of the first group of CRM contents is output.

6. The computer-implemented method of claim 5, wherein
an edit interface is provided for editing a configuration and a layout of the text and the at least one parameter.

7. The computer-implemented method of claim 1, wherein,
when a record of medical consultation of the recipient at the hospital is confirmed within a predetermined period calculated backwards from a time of the transmitting, an application for telemedicine provided by the medical institution client is provided being included in a CRM content.

8. The computer-implemented method of claim 1, wherein
in response to a record of medical consultation of the recipient at the hospital within a predetermined period calculated backwards corresponding to a date and time among the dates and times, an application for telemedicine provided by the medical institution client is provided being included in a CRM content.

9. (canceled)

10. The computer-implemented method of claim 1, wherein
the deletion interworks to change a medical appointment of EMR software used in the hospital.

11. A non-transitory computer-readable storage medium storing instructions that, when executed by a processor, cause a processor to perform the method of claim 1.

12. An electronic device comprising:
one or more processors;
a communication interface operatively coupled to the one or more processors and configured to provide communication with the medical institution client and the terminal of the recipient; and
a storage configured to store a customer relationship management (CRM) content,
wherein the one or more processors are configured to perform the method of claim 1.
